(19) United States
(12) Patent Application Publication
(10) Pub. No.: US 2005/0152862 A1
(43) Pub. Date: Jul. 14, 2005

(54) Cosmetic peeling method

(75) Inventors: Gabrielle Sore, Paris (FR); Isabelle Hansenne, Westfield, NJ (US)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/967,233

(22) Filed: Oct. 19, 2004

Related U.S. Application Data
(60) Provisional application No. 60/530,898, filed on Dec. 22, 2003.

(30) Foreign Application Priority Data
Oct. 29, 2003 (FR) 0350742

(51) Int. Cl. A61K 7/06

(52) U.S. Cl. 424/70.1

(57) ABSTRACT

The present invention relates to a cosmetic peeling method using a composition containing: (a) from 10 to 70% by weight of at least one compound chosen from: nicotinic acid, nicotinic acid esters and nicotinamide, and (b) from 10 to 70% by weight of at least one compound chosen from: ascorbic acid and its precursors, each relative to the total weight of the composition. It also relates to the use of the abovementioned composition for performing a superficial chemical peeling by topical application to the skin, in order to attenuate wrinkles and fine lines and/or pigmented spots and/or scars such as acne or varicella marks.

### REFERENCE TO PRIOR APPLICATIONS

This application claims priority to U.S. provisional application 60/530,898 filed Dec. 22, 2003, and to French patent application 0350742 filed Oct. 29, 2003, both incorporated herein by reference.

### FIELD OF THE INVENTION

The present invention relates to a cosmetic peeling method comprising the topical application, to the skin, of a composition comprising: (a) from 10 to 70% by weight of at least one compound chosen from: nicotinic acid, nicotinic acid esters and nicotinamide (also referred to as niacinamide), relative to the total weight of the composition, and (b) from 10 to 70% by weight of at least one compound chosen from: ascorbic acid and its precursors, relative to the total weight of the composition.

It also relates to the use of the abovementioned composition for performing a superficial chemical peeling by topical application to the skin, in order to attenuate wrinkles and fine lines and/or pigmented spots and/or scars such as acne or varicella marks.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Nicotinic acid and nicotinamide are enzyme cofactors grouped under the term of “vitamin B3” or “vitamin PP”. In addition to its nutritional benefit, which is exploited in the food sector, vitamin B3 has in particular vasodilatory, keratolytic, antiseborrheic, anti-inflammatory and epidermal differentiation and dermal proliferation regulating properties, and depigmenting properties, which make it an active agent of choice in the cosmetic field.

For its part, ascorbic acid, or vitamin C, possesses numerous virtues which can be used in cosmetics, and in particular anti-free radical, depigmenting and anti-aging properties—by stimulating the biosynthesis of various forms of epidermal collagen, laminin, proteoglycans and ceramides and inhibition of matrix metalloproteinases.

Vitamin C has moreover been described as a possible constituent, as a lightening agent, of an exfoliating composition which may moreover include nicotinamide as melanogenesis inhibitor (U.S. Pat. No. 5,874,463) and including, as essential constituent, at least one compound chosen from salicylic acid, retinoic acid, pyruvic acid, resorcinol and glycolic acid. In this document, it is used as a minor constituent of a salicylic acid-based superficial peeling.

Chemical peelings are a well-known means for improving the surface appearance of the skin, in particular for attenuating pigmentation defects such as actinic lentigo or acne or varicella marks, or for smoothing the irregularities of the skin texture, in particular wrinkles and fine lines, by causing a limited destruction of the epidermis and of the superficial layers of the dermis.

Although the compositions described in patent U.S. Pat. No. 5,874,463 have been able to give satisfactory results, it is nevertheless the case that they are not free of side effects. Thus, peelings with salicylic acid can give rise to cases of salicylism in the event of an overdose or a prolonged application. In addition, retinoic acid is associated with risks of teratogenesis and it increases the sensitivity of the skin towards UV radiation.

The need therefore remains to have peeling compositions which are effective while being well tolerated.

### DETAILED DESCRIPTION OF THE PREFERRED EMBODIMENTS

The inventors have discovered, among other things, that vitamins B3 and C, and/or their precursors, combined in substantial concentrations in a topical composition make it possible to perform effective and well-tolerated chemical peelings, without it being necessary to add other exfoliating active agents thereto.

There have of course already been described in patent U.S. Pat. No. 6,071,888 compositions, which may be used by the topical route, comprising from 1 to 20% by weight of nicotinic acid (or its precursors) and from 10 to 30% by weight of ascorbic acid. These compositions are intended for the treatment of melanomas by stimulating the immune response. It is not envisaged that these compositions are applied to skin with visible or tactile skin imperfections, and then rinsed in order to perform a peeling intended to attenuate these imperfections.

One subject of the present invention is therefore a cosmetic peeling method comprising the topical application, to the skin, of a composition comprising, in a physiologically acceptable medium: (a) from 10 to 70% by weight of at least one compound chosen from: nicotinic acid, nicotinic acid esters and nicotinamide, and (b) from 10 to 70% by weight of at least one compound chosen from: ascorbic acid and its precursors, each relative to the total weight of the composition. Preferably, and to the extent necessary for a chemical peel composition, the composition is suitable for topical application to the skin.

It also relates to the use of this composition for performing a superficial chemical peeling by topical application to the skin, in order to attenuate wrinkles and fine lines and/or pigmented spots and/or scars such as acne or varicella marks. For this purpose the invention composition may optionally be formulated as, or used in the manufacture of, a dermatological preparation.

The first constituent of the composition according to the invention is at least one compound selected form the group consisting of nicotinic acid, its esters, and nicotinamide. Useful nicotinic acid esters which can be used in the present invention include alkyl esters of nicotinic acid and of a C₁-C₂₀ alcohol, chosen for example from methyl, ethyl, butyl, hexyl, octyl, decyl, dodecyl and myristyl alcohols. Nicotinamide is, however, preferred for use in the present invention.

The second constituent of the invention composition is at least one compound selected form the group consisting of ascorbic acid and its precursors. Useful precursors of ascorbic acid include ascorbyl palmitate, ascorbyl stearate, magnesium ascorbyl phosphate, sodium ascorbyl phosphate, disodium ascorbyl sulphate, sulphinyl-L-ascorbic acid and glucopyranosyl-L-ascorbic acid. Ascorbic acid is preferred for use in the present invention.

The quantity of each of the compounds (a) and (b) according to the invention represents from 10 to 70% by weight of the composition. Preferably, the quantity of compound (a) varies from 10 to 50% of the weight of the composition and, even better, from 10 to 40% of the weight of the composition, including 15, 20, 25, 30, 35, 45, 55, 60 and 65%. The quantity of compound (b) preferably varies from 10 to 50% of the weight of the composition and, even better, from 10 to 40% of the weight of the composition, including 15, 20, 25, 30, 35, 45, 55, 60 and 65%. Those skilled in the art will know how to adjust these quantities according to the depth of the peeling which has to be performed, that is to say the layers of epidermis or of dermis which have to be removed, in view of this disclosure.

The composition according to the invention is preferably suitable for topical application to the skin and therefore generally comprises a physiologically acceptable medium, that is to say one which is compatible with the skin and optionally with its superficial body growths.

It may be provided in any form, including all the galenic forms conventionally used for topical application. Preferably, the invention composition can be easily removed by rinsing, and in particular is provided in the form of an aqueous gel or of an aqueous or aqueous-alcoholic solution. It may also, by adding a fatty or oily phase, be provided in the form of a lotion-type dispersion or of an emulsion of liquid or semiliquid consistency, preferably obtained by dispersing a fatty phase in an aqueous phase (OW). These compositions may be prepared according to customary methods.

According to a preferred embodiment of the invention, the composition contains a continuous aqueous phase.

When the composition is in the form of an emulsion, the proportion of the oily phase of the emulsion is not limited and may range for example from 1 to 30% by weight, and preferably from 5 to 20% by weight relative to the total weight of the composition. The oils, emulsifiers and coemulsifiers used in the composition in the form of an emulsion may be chosen from those used in the cosmetic and/or dermatological field. The emulsifier and coemulsifier can generally be present in the composition in a proportion ranging from 0.3 to 30% by weight, and preferably from 0.5 to 20% by weight relative to the total weight of the composition. The emulsion may additionally contain lipid vesicles.

As fatty substances which can be used in the invention, it is possible to use oils and in particular mineral oils (liquid paraffin), oils of plant origin (avocado oil, soya bean oil), synthetic oils (perhydrosqualene), silicone oils (cyclomethicone) and fluorinated oils (perfluoropolyethers).

As emulsifiers and coemulsifiers which can be used in the invention, there may be mentioned for example fatty acid esters of polyethylene glycol such as PEG-100 stearate, PEG-50 stearate and PEG-40 stearate; fatty acid esters of polyols such as glyceryl stearate, sorbitan tristearate and the oxyethylenated sorbitan stearates available under the trade names Tween® 20 or Tween® 60, for example; and mixtures thereof.

The composition according to the invention may also contain adjuvants known now and discovered in the future that are useful in the cosmetic and dermatological fields, such as thickeners, active agents, preservatives, solvents and fillers. The quantities of these various adjuvants may be those conventionally used in the fields considered, and are for example from 0.01 to 20% of the total weight of the composition. The adjuvants, depending on their nature, may be introduced into the fatty phase or into the aqueous phase. These adjuvants, and their concentrations, should be such that they do not adversely affect the properties of the compounds according to the invention.

Useful thickeners include in particular: xanthan gum, an optionally crosslinked acrylic acid homo- or copolymer, a polyacrylamide, an acrylamidomethylpropanesulphonic acid homo- or copolymer, and the cellulose derivatives including hydroxypropylcellulose.

The composition of the invention advantageously further comprises at least one compound chosen from alcohols and polyols comprising from 1 to 3 carbon atoms. As examples of such compounds, there may be mentioned ethanol, isopropanol, glycerol and propylene glycol.

In addition, according to a preferred embodiment of the invention, the composition does not contain retinoic acid or glycolic acid or resorcinol, or pyruvic acid or salicylic acid, or chitosan.

As indicated above, this composition according to the invention is intended to be used for performing a chemical peeling, preferably superficial, and may be used to attenuate wrinkles and fine lines and/or pigmented spots and/or scars. The composition is preferably used on people with acne and/or wrinkles and/or scars and/or pigmentation defects such as melasmas and senile or actinic lentigo. It may be applied to the face and/or the neck and/or the neck and shoulders and/or the hands and/or the back, or anywhere else on the skin or scalp in need thereof.

The method according to the invention preferably comprises:
(a) topically applying, to an area of human skin, a composition as defined above,
(b) leaving the composition in contact with the skin for a period of between 5 min and 6 hours, and
(c) removing the composition, for example by rinsing.

The composition used according to the invention may be applied by any means, and is preferably applied using a means allowing uniform distribution and in particular with the aid of cotton wool, a stick, a brush, a gauze, a spatula or a pad, or alternatively by spraying, and may be removed by rinsing with water or with the aid of a mild detergent after having been left in contact with the skin.

To optimize its effects, the peeling method preferably comprises additional steps of preparing the skin for peeling (in order to improve the efficiency and homogeneity of the peeling) and/or of treating the skin after peeling with the aid of compositions containing smaller quantities of vitamin B3 and/or vitamin C than the composition described above.

The compositions used in these preliminary and additional steps may be applied in the morning and the evening, for example optionally in combination with a composition intended to protect the skin against the effects of UV radiation. The pretreatment composition may be applied for one to four weeks and the posttreatment composition for one day to eight weeks, for example.

The above peeling method, including the optional preliminary and additional steps, may be carried out once or repeated if necessary or desired, for example up to five times or more, the peeling sessions being preferably one to eight weeks apart.

The invention will now be illustrated by the following nonlimiting examples. In these examples, the quantities are indicated as a percentage by weight.

### EXAMPLES

The invention will now be illustrated by the following non-limiting examples. In these examples, the amounts are indicated as percentages by weight, unless otherwise indicated.

### Example 1

### Anti-Wrinkle Peeling Composition

The composition below is prepared:

| | |
|---|---|
| Nicotinamide | 10% |
| Ascorbic acid | 10% |
| Glycerine | 10% |
| Purified water | qs 100% |

This composition may be applied in the form of a peeling in order to attenuate facial wrinkles and fine lines.

### Example 2

### Lightening Peeling Composition

The composition below is prepared:

| | |
|---|---|
| Nicotinamide | 15% |
| Ascorbic acid | 15% |
| Calcium pantetheinesulphonate | 5% |
| Propylene glycol | 30% |
| Purified water | qs 100% |

This composition may be applied in the form of a peeling in order to attenuate dyschromia (actinic or senile lentigo, melasma).

### Example 3

### Anti-Acne Peeling Composition

The composition below is prepared:

| | |
|---|---|
| Nicotinamide | 12% |
| Ascorbic acid | 12% |
| Hydroxypropylcellulose | 2% |
| Ethanol | 30% |
| Purified water | qs 100% |

This composition may be applied in the form of a peeling in order to attenuate acne marks.

### Example 4

### Cosmetic Peeling Composition

The composition below is prepared:

| | |
|---|---|
| Nicotinic acid | 10% |
| Ascorbic acid | 10% |
| Glycerine | 10% |
| Purified water | qs 100% |

### Example 5

### Anti-Wrinkle Peeling Composition

The composition below is prepared:

| | |
|---|---|
| Myristyl nicotinic acid ester | 10% |
| Ascorbic acid | 10% |
| Glycerine | 10% |
| Purified water | qs 100% |

The above written description of the invention provides a manner and process of making and using it such that any person skilled in this art is enabled to make and use the same, this enablement being provided in particular for the subject matter of the appended claims, which make up a part of the original description and including a peeling method, comprising the topical application to the skin of a person in need thereof, of a composition comprising:
(a) from 10 to 70% by weight relative to the total weight of the composition of at least one compound selected from the group consisting of nicotinic acid, nicotinic acid esters, and nicotinamide, and
(b) from 10 to 70% by weight relative to the total weight of the composition of at least one compound selected from the group consisting of ascorbic acid and ascorbic acid precursors.

The invention method of peeling is preferably a peeling intended to attenuate wrinkles and/or fine lines and/or pigmented spots and/or scars, including peeling to attenuate acne or varicella marks, and peeling to attenuate acne and/or wrinkles and/or scars and/or pigmentation defects. Thus, the invention composition is preferably applied to areas of skin in need of such treatment in order to bring about the desired results. Application can include application to the face and/or the neck and/or the shoulders and/or the hands and/or the back.

The compositions of the invention are preferably formulated such that they are effective for their intended purpose(s); thus they preferably comprise effective amounts of the noted components, considered in view of both the concentration/strength of the composition and the time of application to the skin, to provide the noted benefits.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A peeling method, comprising the topical application to the skin of a person in need thereof, of an effective amount of a composition comprising:
(a) from 10 to 70% by weight relative to the total weight of the composition of at least one compound selected from the group consisting of nicotinic acid, nicotinic acid esters, and nicotinamide, and
(b) from 10 to 70% by weight relative to the total weight of the composition of at least one compound selected from the group consisting of ascorbic acid and ascorbic acid precursors.

2. The method according to claim 1, comprising at least one nicotinic acid ester selected from the group consisting of alkyl esters of nicotinic acid and a C₁-C₂₀ alcohol.

3. The method according to claim 2, comprising at least one nicotinic acid ester selected from the group consisting of methyl, ethyl, butyl, hexyl, octyl, decyl, dodecyl and myristyl nicotinic acid esters.

4. The method according to claim 1, comprising at least one ascorbic acid precursor selected from the group consisting of ascorbyl palmitate, ascorbyl stearate, magnesium ascorbyl phosphate, sodium ascorbyl phosphate, disodium ascorbyl sulphate, sulphinyl-L-ascorbic acid and glucopyranosyl-L-ascorbic acid.

5. The method according to claim 1, wherein the quantity of compound (a) represents from 10 to 50% of the total weight of the composition.

6. The method according to claim 1, wherein the quantity of compound (b) represents from 10 to 50% of the total weight of the composition.

7. The method according to claim 1, wherein the composition does not comprise retinoic acid or glycolic acid or resorcinol, or pyruvic acid or salicylic acid or chitosan.

8. The method according to claim 1, wherein said method consists of:
(a) topically applying, to an area of human skin in need thereof, said composition,
(b) leaving the composition in contact with said skin for a period of between 5 min and 6 hours, and
(c) removing the composition.

9. The method according to claim 8, wherein the composition is left in contact with the skin in step (b) for a period of between 5 min and 30 min.

10. The method according to claim 1, wherein the composition is applied to the face and/or the neck and/or the shoulders and/or the hands and/or the back.

11. The method according to claim 5, wherein the quantity of compound (b) represents from 10 to 50% of the total weight of the composition.

12. The method according to claim 1, wherein said peeling is a peeling to attenuate wrinkles and fine lines and/or pigmented spots and/or scars.

13. The method according to claim 11, wherein said peeling is a peeling to attenuate acne or varicella marks.

14. The method according to claim 1, wherein said peeling is a peeling to attenuate acne and/or wrinkles and/or scars and/or pigmentation defects.

15. The method according to claim 1, wherein said nicotinic acid esters are selected from the group consisting of alkyl esters of nicotinic acid and a C₁-C₂₀ alcohol, and said ascorbic acid precursors are selected from the group consisting of ascorbyl palmitate, ascorbyl stearate, magnesium ascorbyl phosphate, sodium ascorbyl phosphate, disodium ascorbyl sulphate, sulphinyl-L-ascorbic acid and glucopyranosyl-L-ascorbic acid.

16. The method according to claim 15, wherein the quantity of compound (a) represents from 10 to 50% of the total weight of the composition, and the quantity of compound (b) represents from 10 to 50% of the total weight of the composition.

17. The method according to claim 15, wherein the composition does not comprise retinoic acid or glycolic acid or resorcinol, or pyruvic acid or salicylic acid or chitosan.

18. The method according to claim 15, wherein said method consists of:
(a) topically applying, to an area of human skin in need thereof, said composition,
(b) leaving the composition in contact with said skin for a period of between 5 min and 6 hours, and
(c) removing the composition.

19. The method according to claim 18, wherein the composition is left in contact with the skin in step (b) for a period of between 5 min and 30 min.

20. The method according to claim 15, wherein the composition is applied to the face and/or the neck and/or the shoulders and/or the hands and/or the back, and wherein said peeling is a peeling to attenuate acne and/or wrinkles and/or fine lines and/or pigmentation defects and/or scars.

21. The method according to claim 1, wherein said composition comprises ascorbic acid.

22. The method according to claim 1, wherein said composition comprises nicotinamide.

23. The method according to claim 1, wherein said composition comprises nicotinamide and ascorbic acid.
